(19) United States
(12) Patent Application Publication
(10) Pub. No.: US 2021/0131996 A1
(43) Pub. Date: May 6, 2021

(54) BIOMARKER SENSOR APPARATUS AND METHOD OF MEASURING BIOMARKER IN BLOOD

(71) Applicant: BAILRIGG DIAGNOSTICS LIMITED, Lancaster, Lancashire (GB)

(72) Inventors: Mukesh KUMAR, Lancaster (GB); Peter Robert FIELDEN, Rossendale (GB)

(21) Appl. No.: 16/629,475

(22) PCT Filed: Jul. 10, 2018
(86) PCT No.: PCT/GB2018/051948

§ 371 Date: Jan. 8, 2020

(30) Foreign Application Priority Data
Jul. 10, 2017 (GB) 1711051.1
Apr. 16, 2018 (GB) 1806187.9

(51) Int. Cl. G01N 27/327; B01L 3/00; C12Q 1/00; G01N 33/49

(52) U.S. Cl. CPC G01N 27/3274; B01L 3/502715; B01L 3/502753; C12Q 1/005; B01L 2300/126; G01N 27/3272; B01L 2200/10; B01L 2300/0645; B01L 2200/0647; G01N 33/491

(57) ABSTRACT

The present invention relates to a device for measuring a biomarker, in particular lactate, in a sample consisting of whole blood. The device includes at least one pre-determined amount of the biomarker. Generally, the device includes a porous separation means to separate whole blood into its constituent parts. There is also provided a method of measuring a biomarker in whole blood.

### BACKGROUND

### Technical Field

The present invention relates to a lactate sensor system and a method of measuring lactate in blood.

### Description of Related Art

Measurement of lactate levels in blood is an important measure in critical healthcare. For example, blood lactate monitoring is used as an indirect marker of tissue hypoxia. Increased lactate levels may reflect increased morbidity and high mortality. The use of blood lactate monitoring has a place in risk-stratification in critically ill patients.

Furthermore, the measurement of lactate in the blood of healthy individuals is also advantageous. Lactate is a fitness marker and blood lactate is measured in the sports industry.

Lactate is a by-product produced in the body during normal metabolism and exercise. Blood lactate levels serve as an indirect marker for biochemical events such as fatigue within exercising muscle. Athletes may use lactate levels to track their training progress. Indeed, any professional activities that require high physical loading involving muscle strain and/or mechanical work may benefit from the measurement of blood lactate level.

Blood lactate measurements are thus advantageous at point-of-care and for routine wellbeing assessment.

There are known methods for measuring lactate in blood. Currently, measurements are usually carried out in a laboratory, resulting in a delay between the taking of the blood sample and return of the lactate concentration. Analysers used in a laboratory may require up to 3 mL of blood per assay.

In a laboratory environment, lactate may be measured by a two-stage reaction where lactate is first converted to pyruvate through a bio-catalytic reaction with lactate oxidase that generates hydrogen peroxide as a by-product. The hydrogen peroxide is subsequently measured using a second bio-catalyst, such as peroxidise, which causes a spectrophotometric colour change which is monitored and calibrated to derive the quantity of the lactate in the blood sample. There is a need for a rapid method of lactate measurement in blood samples at point-of-care and for personal well-being assessment. A delay reduced to less than 10 minutes, and a test that requires less than 100 μL of blood, present many advantages.

Amperometric biosensors are known for their specificity and simplicity of assembly. Such biosensors based on electrodes that have been fabricated by screen-printing techniques are widespread for the routine personal care measurement of glucose in blood for diabetic patients.

More recently, screen-printed amperometric biosensors that measure lactate in blood serum have been reported in the academic literature. However, to date there has been no development of sensors that monitor lactate in whole blood as a point-of-care device.

### SUMMARY OF THE INVENTION

According to the present invention there is provided a lactate sensor apparatus and a method of measuring lactate in blood.

According to one aspect of the present invention, there is provided a device for measuring at least one biomarker in a biological fluid (generally consisting of whole blood), comprising:

an entrance for the biological fluid;

at least one measurement cell, each measurement cell comprising three electrodes, wherein at least one electrode per measurement cell comprises a bio-catalyst specific to the at least one biomarker and at least one electrode per measurement cell comprises an electrochemical mediator, a separation means provided between the entrance and the measurement cells wherein the separation means is suitable to separate the biological fluid into its constituent components and is suitable to transport at least one of the biological fluid components towards the measurement cells.

Generally, the device includes at least two measurement cells, suitably two or more measurement cells.

Generally, a first region of the separation means includes a first pre-determined amount of the biomarker, in particular lactate, proximate to one of the measurement cells, wherein the first region is not proximate to any of the other measurement cells. Upon transfer of the biological fluid component(s) towards the measurement cell, the biological fluid component mixes with the pre-determined amount of biomarker. The first pre-determined amount of biomarker does not generally mix with the biological fluid prior to separation thereof.

The inclusion of the pre-determined amount(s) of the biomarker is useful in calibrating the device. The device of the present invention generally includes an indication of the pre-determined amount(s) of the biomarker. The inclusion of a portion of standard pre determined biomarker, in particular lactate, in one of the two branches that delivers the separated blood plasma to two measurement cells gives a simultaneous reading of (unknown lactate concentration) in one branch and (unknown lactate concentration+X), where X is the pre-determined amounts of the biomarker added (from the pre-determined standard (in this case lactate), to the unknown amount. This gives two points on a straight line calibration, where the line is extrapolated back to the equivalent of zero concentration, thus yielding a negative concentration that is identical to the unknown concentration.

Another possibility is to add a third branch where the pre-determined amount is, for instance, quantitatively 2×. This then gives the same straight line and calibration process, but it uses three rather than two points to define the straight line—so offering better precision.

The advantage of the calibration is in situ and in real time and simultaneous with the detection of the unknown concentration of the lactate is the common mode rejection of many undesirable factors, including ageing of the enzyme and/or the mediator (Meldola blue in this example). It also corrects for temperature effects on the calibration process.

The in situ calibration is helpful to overcome the gradual deterioration of the enzyme. This ultimately gives better shelf-life, not because the enzyme last longer, but because the deterioration is measured and there by corrected.

Devices to measure biomarkers such as lactate are generally manufactured at least several months before use. During storage, calibration of known devices can drift and the accuracy of the device can deteriorate over this time. The device of the present invention allows calibration immediately prior to use. The calibration check itself is very quick, generally taking 5 minutes or less, typically 1 minute or less. The calibration check is also suitable for use by users without medical training as it is self-contained within the device, requiring no external intervention or dispensing of reagents or standards, which is also particularly useful for home users.

In this context, a region proximate to one of the measurement cells, generally refers to a region within the 10% of the length of the separation means closest to the measurement cell wherein the region is at a distance of more than 10% of the length of the separation means from any of the other measurement cells (suitably more than 15% of the length).

Additionally, or alternatively, the term “proximate” is generally used to refer to a distance around 5 mm or less from the portion of the measurement cell nearest to the separation means, typically 3 mm or less, suitably 1 mm or less.

According to a further aspect of the present invention, there is provided a device for measuring a biomarker in a sample consisting of whole blood, comprising:

an entrance for whole blood;

two or more measurement cells, each measurement cell comprising three electrodes, wherein at least one electrode per measurement cell comprises a bio-catalyst specific to the biomarker and at least one electrode per measurement cell comprises an electrochemical mediator;

a separation means provided between the entrance and the measurement cells wherein the separation means is suitable to separate whole blood into blood components and is suitable to transport at least one of the blood component(s) towards the measurement cells;

and wherein the separation means includes a first pre-determined amount of the biomarker at a region proximate to one of the measurement cells, wherein the region is within the 10% of the length of the separation means closest to the measurement cell and wherein the region is at a distance of more than 15% of the length of the separation means from any of the other measurement cells.

The device of the present invention generally includes an indication of the pre-determined amount(s) of the biomarker. This may, for instance be included on the packaging of the device or may be in the form of a chip included in the device.

According to one embodiment, a second region of the separation means includes a second pre-determined amount of the biomarker, in particular lactate, proximate to one of the measurement cells, wherein the second region is not proximate to any of the other measurement cells. Upon transfer of the biological fluid component(s) towards the measurement cell, the biological fluid component mixes with the pre-determined amount of biomarker. The second pre-determined amount of biomarker does not generally mix with the biological fluid prior to separation thereof. This is useful in calibrating the device. The first and second pre-determined amounts of biomarker are different.

Generally, the region proximate to the measurement cell is spaced away from the entrance and the unseparated biological fluid sample (generally whole blood sample) does not contact the first or second pre-determined amounts of lactate.

Alternatively, the second measurement cell may include a first pre-determined amount of biomarker (generally lactate).

According to one embodiment, the device comprises at least three measurement cells wherein the third measurement cell includes a second pre-determined amount of biomarker (generally lactate) at a region proximate to one of the measurement cells and the first pre-determined amount differs from the second pre-determined amount. The region is within the 10% of the length of the separation means closest to the measurement cell and wherein the region is at a distance of more than 15% of the length of the separation means from any of the other measurement cells.

The region proximate to the measurement cell is generally spaced away from the entrance by a distance corresponding to more than 10% of the length of the separation means and the whole blood sample does not contact the first pre-determined amount of the biomarker or the second pre-determined amount of the biomarker.

Typically, the separation means extends from the entrance to the measurement cell(s), or to within 5 mm of each of the measurement cells, generally to within 3 mm of each of the measurement cells, suitably to within 1 mm of each of the measurement cells.

According to one embodiment, the device includes four measurement cells, wherein the third measurement cell includes a bio-catalyst specific to a second bio-marker and the fourth measurement cell includes a bio-catalyst specific to the second biomarker and a pre-determined amount of the second biomarker. Alternatively, a region of the separation means proximate to the fourth measurement cell may include a pre-determined amount of the second biomarker

According to a further aspect of the present invention there is provided a method of measuring a biomarker in a biological fluid comprising:

providing the device as described herein;

introducing a biological fluid sample (generally a whole blood sample) through the entrance of the device;

allowing the separation means to separate the biological fluid sample into its components;

obtaining a measurement from the measurement cells;

wherein the measurement is obtained within 5 minutes of the sample being introduced through the entrance.

As noted above, the device of the present invention includes a pre-determined amount of the biomarker in one of two branches or pathways that delivers the separated blood (blood plasma) to the measurement cells. A measurement from one of the branches or pathways equates to a reading of the biomarker in the biological fluid sample [unknown biomarker concentration]. A measurement from the other of the branches or pathways equates to a reading of the reading of the biomarker in the biological fluid combined with a reading from the pre-determined amount of the biomarker [unknown biomarker concentration+X (where X=the pre-determined amount of the biomarker)].

The method of the present invention thus provides two points on a straight line calibration. The line can be extrapolated back to the equivalent of a zero concentration, thus yielding a negative concentration that is identical to the unknown concentration. This calibration is delivered in situ within the structure and geometry of the measurement device by virtue of the transport properties of the separation strips being able to mix the pre-loaded biomarker (generally lactate) with the biomarker already in the blood plasma.

According to one embodiment, the method may include a second pre-determined amount of the biomarker, providing a third point on the straight line calibration. For instance, the second pre-determined amount may be double or half of the first pre-determined amount.

Generally, measurements are made simultaneously from the measurement cells. Suitably, multiple measurements are made from each measurement cell at precisely selected time intervals.

According to a further aspect of the present invention there is provided a method of diagnosing a disease or condition in an individual including measuring the levels of the biomarker(s) identified herein in a biological fluid sample from an individual, comparing the level of the identified biomarker(s) with control data relating to the same biomarker(s) in the same type of biological fluid sample wherein if the level of the identified biomarker(s) is increased or reduced by 10% or more compared to the control data, the individual is diagnosed with the disease or condition. Generally, the level of the identified biomarker(s) is increased by 10% or more compared to the control data and the condition is tissue hypoxia.

Generally, the biological fluid is blood, the biological fluid component is blood serum and the biomarker is lactate.

The device comprises at least two measurement cells, for example screen-printed electrode measurement cells, each comprising a three electrode measurement cell (carbon working electrode; carbon counter electrode; and silver/silver chloride reference electrode) pre-coated with two essential reagents: a bio-catalyst and an electrochemical mediator.

The bio-catalyst is specific to the biomarker of interest.

One electrode cell measures the biomarker concentration from the biological fluid sample. Generally, the other electrode cell, or a region of the separation means proximate thereto, includes a predetermined amount of the biomarker and this electrode measures the arithmetic sum of the biomarker concentration from the biological fluid sample and the predetermined amount of biomarker. This provides an in situ standard addition measurement to facilitate common-mode rejection and internal calibration.

The device also includes a separation means suitable to separate the biological fluid into its constituent components, generally based upon capillary action and passive diffusion of the biological fluid (generally whole blood) from the entrance, to the two electrode measurement system via the separation means. The transport of the biological fluid and its constituent component(s) of interest are monitored to ensure optimal interaction with both electrochemical measurement cells, and the assay is initiated on complete transport of the constituent component(s) of interest to the measurement cells.

According to one embodiment, the biological fluid is whole blood, the constituent component of interest is blood serum and the biomarker is lactate. Generally, red blood cells are separated from the whole blood by the separation means and are not transported to the measurement cells. Generally, the biological fluid is from an animal including a human. However, the systems and methods may also be used for other animals, and mention may be made of livestock such as horses, cows, sheep, pigs and camels and of pets such as dogs, cats and rabbits.

According to one embodiment, the present invention provides a device for measuring lactate within a small volume sample of whole blood (typically as little as 50 μL of whole blood). The present invention may include measuring more than one biomarker in a biological fluid. In such embodiments, the device includes measurement cells comprising bio-catalysts specific to each biomarker to be measured.

In the methods of the present teachings, the levels of biomarkers can be determined by a variety of techniques known in the art, for example, electroanalytical techniques, preferably chrono-amperometry.

The teachings of the present invention, which in some embodiments may be implemented by a processing device or system implements a method that includes obtaining a set of biological fluid sampling data for an individual.

The methods can include transmitting, displaying, storing, or printing; or outputting to a user interface device, a computer readable storage medium, a local computer system or a remote computer system, information related to the presence and amount of the identified biomarker(s) in the sample. Various features and steps of the methods of the present teachings can be carried out with or assisted by a suitably programmed computer, specifically designed and/or structured to do so.

The method of the present invention may include accessing a control data set that includes a control level for the or each of the biomarkers assessed in a sample of the same type of biological fluid, and comparing the measured levels of biomarkers with the control levels to determine whether the amount of the individual's biomarkers in the sample is elevated compared to the control level.

The method may include using the determined number and/or level of the biomarkers compared to the control data to assign a probability that the individual should be classified as suffering from tissue hypoxia.

The sample can include or consist of a biological fluid. Particular mention may be made of sputum, serum, blood, urine and cerebrospinal fluid. Generally, the biological fluid is whole blood.

The device of the present invention is generally an amperometric biosensor. According to an aspect of the present invention there is provided a chrono-amperometric measurement protocol that makes multiple measurements of both electrode systems simultaneously and at precisely selected time intervals to gather optimum electroanalytical data. These data are then processed by an algorithm that rejects common mode artefacts, compensates for ageing effects of the bio-catalysts, and introduces in situ calibration by providing both the absolute lactate concentration in the blood sample, and also a quality parameter that validates the lactate measurement.

As is discussed in more detail below, the example embodiments address many of the difficulties of the related art and provide a mechanism for measuring lactate in small volumes of whole blood within a short time.

An example embodiment provides the further advantages of compact size, low power consumption and portability, making it suitable for point-of-care measurements.

In one example, cathodic measurements may be performed at a screen-printed carbon electrode mediated with the electron transfer reagent Meldola's Blue in conjunction with the oxidised form of the cofactor nicotinamide adenine dinucleotide (NAD+) and in the presence of the enzyme lactate dehydrogenase. Monitored at a single working electrode, such a combination provides the means for the quantitative determination of lactate in aqueous solution. [1] Surprisingly, the pre-addition of a pH controlling buffer and the inclusion of a porous separation mean, such as chemically-modified filter paper or a paper composite material with similar transport properties, may facilitate the direct measurement of lactate in human blood serum.

The separation means generally includes pores, wherein at least 95% of the pores may have a pore size diameter of from about 2 μm to about 10 μm; suitably of from about 5 μm to about 10 μm; typically, of from about 7 μm to about 10 μm.

According to one embodiment, the mean pore size diameter of the separation means is from about 5 to about 9 μm.

The separation means generally comprises paper, such as filter paper. Typically, the separation means comprises cellulose paper, for instance cellulose filter paper which may be chemically modified, for example with reagents such as octadecyltrichlorosilane, diphenyldichlorosilane, cyclohexyl isocyanate and phenyl isocyanate ethylenediaminetetraacetic acid (EDTA), EDTA dianhydride filter paper).

Generally the separation means includes or comprises a composite of two materials having different porosity, generally two fibrous materials having different porosities.

Typically, the separation means includes a paper composite comprising a material with a lower porosity than the paper, such as a material comprising silica fiber. According to one embodiment, the pore size of the paper composite is 2 μm-10 μm (typically wherein the size diameter of the majority of the pores from about 6 μm to about 10 μm),

The separation element may comprise silica fiber. According to one embodiment, the separation means comprises paper and silica fiber.

Whilst the applicant does not wish to be bound by theory, it is believed that the addition of an additional porous material (such as silica fiber) aids in the trapping of red blood cells. This aids in the separation of whole blood into its constituent parts.

The separation means may include more than one layer, typically wherein at least one layer comprises or consists of paper, for instance filter paper, including chemically modified filter paper, and at least one layer comprises or consists of a paper composite including paper and an additional fibrous material, in particular a fibrous material including or consisting of silica.

According to one embodiment, the separation means may be in the form of a composite, comprising paper and silica fiber.

The separation means may include from 30 to 70% paper (generally around 50% paper), and from 30 to 70% paper composite material including silica fiber (generally around 50% paper-silica fiber composite).

In one example, a sequence of chrono-amperometric measurements that follow a strict protocol may be selected to provide an electrical current that is proportional to lactate concentration. Surprisingly, the parameters of: applied potential; current sample time; and scan number may be optimised to improve the reproducibility and repeatability of the measurement. This observation is illustrated in FIG. 5. Even under a sequential measurement scheme, it is found that a precise measurement of lactate may be acquired in under 5 minutes.

In one example, the method may be performed with more than one working electrode according to the descriptions above. Each electrode is subjected to the same liquid sample, but for one electrode the sample remains unchanged, while for each other electrode(s) a deliberate addition of a known quantity of lactate is made to facilitate accurate calibration. Where there are more than two electrodes, the known quantities may differ per electrode. The calibration may follow the known scheme of “standard addition”, or any similar methodology known to the art.

Surprisingly, this method also compensates for any degradation or aging effects of the enzyme, or indeed for any other reagent components.

According to one embodiment, the second measurement cell includes a third pre-determined amount of the biomarker.

According to one embodiment, the device includes at least three measurement cells, wherein the first measurement cell does not include any of the biomarker of interest, the second measurement cell includes a first pre-determined amount of the biomarker of interest, and the third measurement cell includes a second pre-determined amount of the biomarker of interest, wherein the first and second pre-determined amounts are different. According to one embodiment, the third measurement cell includes a fourth pre-determined amount of the biomarker and the first pre-determined amount of the biomarker differs from the fourth pre-determined amount of the biomarker.

Generally, all but one of the measurement cells includes a predetermined amount of the biomarker of interest (generally lactate), where each measurement cell includes a different predetermined amount of the biomarker of interest. Alternatively, all but one of the measurement cells may be proximate to a region of the separation means which includes a predetermined amount of the biomarker of interest.

According to a further embodiment, the device includes at least four measurement cells, wherein the first measurement cell includes a bio-catalyst specific to a first bio-marker, the second measurement cell includes a pre-determined amount of the first biomarker of interest, the third measurement cell includes a bio-catalyst specific to a second bio-marker and the fourth measurement cell includes a pre-determined amount of the second biomarker.

In one example, the measurement sequence is triggered by a conductivity measurement signalling arrival of the blood plasma front at any significant position in the sample transport manifold, for instance at a working electrode.

In another example, target species other than lactate may be measured through a similar regime whereby alternative enzymes, alternative co-factors and/or alternative mediators are used to enable measurements in small volumes of blood.

In another example, several target species may be measured simultaneously through a regime that employs multiple working electrodes, each utilising a specific combination of enzyme, co-factor and mediator for the purpose of enabling a point of care device using a single blood sample to yield quantitative data for multiple targets.

The device of the present invention includes a separation means provided between the entrance and the measurement cells wherein the separation means is suitable to separate the biological fluid into its component parts, and is suitable to transport at least one of the component parts towards the measurement cells.

Generally, the separation means comprises or consists of a paper composite, in particular a paper composite including silica fibers.

According to one embodiment, the mean pore size diameter of the separation means is 2 μm-10 μm, with most pores having a diameter towards the upper end of this range.

The separation means may have an associated thickness of 300 to 500 μm, generally 350 to 450 μm, typically 350 to 400 μm, suitably around 380 μm.

The separation means generally has an associated area of from around 100 to 300 mm²; typically, of from around 150 to 250 mm², suitably of from around 150 to 200 mm². According to one embodiment, the separation means has an associated area of around 189 mm².

The separation means generally has an associated volume of from around 25 to 200 mm³; typically, of from around 50 to 150 mm³, suitably of from around 75 to 150 mm³. According to one embodiment, the separation means has an associated volume of around 70 to 110 mm³.

According to one embodiment, the separation means may include at least one layer of paper and at least one layer of a paper composite including silica fibers.

Typically the outer layer(s) of the separation means comprise or consist of paper, generally the outer layers are formed from paper. Alternatively, the outer layer(s) of the separation means comprise or consist of paper composite, generally the outer layers are formed from paper composite.

Suitably, at least one inner layer of the separation means comprises or consists of a paper composite, in particular a paper composite including silica fibers.

The separation means may comprise one paper layer and one paper composite layer.

Typically, the separation means has an associated volume of from around 1000 to around 4000 mm³ per ml of biological sample, in particular per ml of whole blood sample, generally of from around 1500 to around 3500 mm³ per ml, suitably 1500 to around 300 mm³ per ml.

According to one embodiment, the separation means has an associated volume of from around 1400 to around 2800 mm³ per ml of biological sample, in particular per ml of whole blood sample.

Where the separation means includes a paper composite, for instance a paper composite comprising silica fibers, and the sample is applied to a middle portion of the separation means, the bidirectional flow rate of blood through the separation means is typically 75 sec/50 sample or less; suitably 70 sec/50 μL sample or less where the separation means has an associated area of 150 to 200 mm².

Where the separation means includes a paper composite, for instance a paper composite comprising silica fibers, and the sample is applied to an end portion of the separation means, the unidirectional flow rate of blood through the separation means is typically 150 sec/50 μL sample or less; suitably 130 sec/50 μL sample or less where the separation means has an associated area of 150 to 200 mm².

The separation means generally has an associated porosity of at least 2%, generally 4 to 20%.

Generally, the pores formed within the separation mean have a mean diameter of 5 to 40 μm; typically, 10 to 30 μm, suitably 15 to 25 μm, more suitably 20 to 25 μm.

Suitably, the separation means has an associated porosity of 2%-20%, and/or the pores formed within the separation mean have a mean diameter of 2 to 40 μm.

Typically, at least 30% of the voids are interconnected, generally at least 40%, suitably at least 50%. Suitably the interconnecting portions have a mean diameter of 0.5 to 3 μm.

The separation means generally have an associated thickness of 150 to 250 μm, typically 175 to 225 μm.

The separation means generally have an associated area of around 100 to 300 mm², suitably 150 to 250 mm², typically 150 to 200 mm².

The separation means generally have an associated volume of around 50 to 200 mm³, typically 50 to 150 mm³, suitably 75 to 125 mm³.

Typically, the separation means has an associated volume of around 1500 to 2500 mm³ per ml of biological sample, generally 1750 to 2250 mm³ per ml of biological sample, suitably around 2000 mm³ per ml of biological sample, in particular, per ml of whole blood sample.

At room temperature and pressure, the flow rate of blood through the separation means is typically at least 25 sec/100 mL blood, generally at least 30 sec/100 mL blood, suitably at least 35 sec/100 mL blood.

The separation means generally acts to effect the following
To separate the biological fluid to its constituent components, generally to separate whole blood to blood serum and red blood cells
To transfer one or more of the constituent components of the biological fluid from the entrance to the measurement electrodes, whilst preventing the transfer of at least one of the constituent components from the entrance to the measurement electrodes. Generally the separation means transfers blood serum to the measurement electrodes whilst preventing the transfer of red blood cells and whole blood to the measurement electrodes.
To include the take up of pre-set amounts of the biomarker (generally lactate), previously loaded onto the separation means and then typically dried, that become added to the constituent components (generally, blood plasma) that arrives for measurement at the electrode(s) which facilitates in situ standard addition calibration. Different electrodes will encounter different amounts of added lactate (including zero lactate) to provide data for the calibration procedures described FIGS. 6 and 7.

The separation means is generally in the form of an absorbent strip.

The separation means generally consists essentially or consists of a material having a porosity of at least 2%, suitably at least 3%, typically at least 4%.

The separation means may include more than one layer, generally 2 to 10 layers, suitably 3 to 5 layers. Suitably each layer has an associated porosity of at least 2%, typically at least 4% and/or a pore size of 5 to 40 μm, generally 2 to 10 μm.

Generally each layer is formed from the same material and typically each layer has the same associated porosity, area and volume.

Alternatively, at least one layer may be formed from material different to the other layers, and at least one layer may have different properties, including different porosity.

According to one embodiment, the separation means consists essentially or consists of three to five sheets of material having a porosity of at least 4% and/or a pore size of 2 to 40 μm.

Surprisingly, it is found that the number of layers affects both the sensitivity, precision and assay time of the measurement.

Generally, at least one of the sheets of material from which the separation means is formed is a paper—silica fiber composite.

One or more layers of the separation means may be formed from paper or paper composite with an additional porous material to aid the trapping of red blood cells, in particular paper—silica fiber composite. Alternatively, or additionally, one or more layers of the separation means may be formed from cyclopore polycarbonate membrane.

According to one embodiment, the separation means comprises 3 to 5 layers. Typically, at least some of the layers of the separation means comprise paper (in particular paper—silica fiber composite), generally at least one of the layers of the separation means is formed from a paper—silica fiber composite or cyclopore polycarbonate membrane.

The outer layers of the separation means may be formed from filter paper.

The outer layers of the separation means may have an associated pore size of 20-25 μm. The inner layer(s) may have an associated porosity of 4%-20%.

The outer layers of the separation means may have a greater associated thickness than the inner layer(s). According to one embodiment, the thickness of each of the outer layers is at least 25% greater than the thickness of each of the inner layer(s), suitably around 50% greater.

According to one embodiment, the separation means extends from the entrance to within 5 mm to the/at least one of the measurement cells, typically to within 2 mm of the measurement cells, suitably to the measurement cells.

Suitably the measurement cells of the device described herein may be arranged linearly. Alternatively, the measurement cells may be arranged to radially extend from the entrance.

According to one embodiment, the device comprises one or more conductivity electrode suitable to detect the arrival of liquid sample. A conductivity electrode may be provided at any critical point in the transport geometry to enhance measurement precision and ensure minimisation of assay time.

Generally, the separation means is configured to allow blood serum to be transferred from the entrance to each measurement cell, and the separation means is configured to resist or prevent transfer of red blood cells to the measurement cells.

Suitably, the separation means is configured to transfer blood serum from the entrance to each measurement cell non-vertically, preferably to transfer blood serum from the entrance to each measurement cell horizontally.

According to one embodiment, there is provided a kit including the device disclosed herein and instructions for use. This may include instructions for comparing the level of the biomarkers in the biological fluid sample with a standard or threshold reference score for the same type of biomarker(s) in the same type of biological fluid sample (for instance, blood, sputum, urine etc.).

The foregoing as well as other features and advantages of the present teachings will be more fully understood from the following description, examples and claims.

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, a device or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

As used herein, “reference” or “control” or “standard” each can refer to an amount of a biomarker in a healthy individual or control population or to a risk score derived from one or more biomarkers in a healthy individual or control population. The amount of a biomarker can be determined from a sample of a healthy individual, or can be determined from samples of a control population.

The sources of biological sample types may be different subjects; the same subject at different times; the same subject in different states, e.g., prior to drug treatment and after drug treatment; different sexes; different species, for example, a human and a non-human mammal; and various other permutations. Further, a biological sample type may be treated differently prior to evaluation such as using different work-up protocols.

These and other features and advantages may be appreciated further from the following example embodiments.

### BRIEF DESCRIPTION OF THE DRAWINGS

For a better understanding of the invention, and to show how example embodiments may be carried into effect, reference is now made to the accompanying drawings in which:

FIG. 1 is a perspective view of the disassembled components of an example lactate sensor apparatus.

FIG. 2 is a sectional plan view of the lactate sensor with a single working electrode.

FIG. 3 is a sectional plan view of sensor geometries that have more than one working electrode.

FIG. 4 is a flowchart as a schematic overview of an example method of measuring lactate.

FIG. 4A is a schematic overview of an example method of measuring lactate in blood in terms of operational timing.

FIG. 5 is a graph of typical chrono-amperometric curves for sequential measurements at a single working electrode.

FIG. 6 is a graph of a typical implementation of the calibration method of Standard Addition for a lactate sensor that employs a pair of working electrodes.

FIG. 7 is a graph of a typical implementation of the calibration method of Standard Addition for a lactate sensor that employs multiple working electrodes.

FIG. 8 is a sectional plan view of the lactate sensor where the filter transport element has been extended to incorporate a dual electrode contacting conductivity sensor that monitors the arrival of the blood plasma front.

FIG. 9 is a graph of the geometrical separation of blood plasma from red blood cells is a porous paper—silica fibre composite for different blood volume loadings with unidirectional transport.

FIG. 10 is a graph of the geometrical separation of blood plasma from red blood cells is a porous paper—silica fibre composite for different blood volume loadings with bidirectional transport.

FIG. 11 is a graph of the time taken and distance travelled for separated blood plasma from red blood cells is a porous paper—silica fibre composite for different blood volume loadings with unidirectional transport.

FIG. 12 is a graph of the time taken and distance travelled for separated blood plasma from red blood cells is a porous paper—silica fibre composite for different blood volume loadings with bidirectional transport.

### DETAILED DESCRIPTION OF THE EXAMPLE EMBODIMENTS

The example embodiments are described with reference to a lactate sensor apparatus and method. The example embodiments described below relate to the measurement of lactate. In other embodiments, other blood-borne species of clinical and healthcare significance may be measured with an appropriate selection of the enzyme, cofactor and mediator chemistries. The apparatus and method may be applied in many specific implementations, as is apparent to persons skilled in the art from the teachings herein.

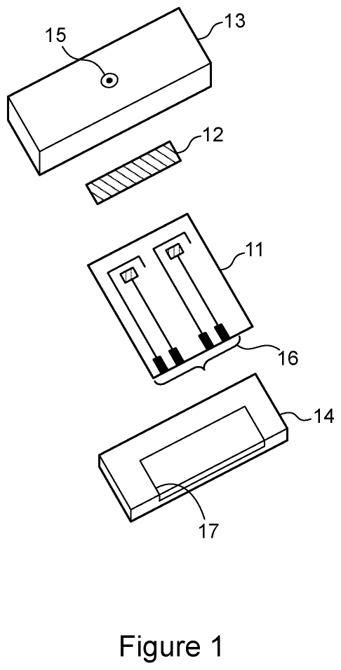

FIG. 1 is a perspective view of an example lactate sensor apparatus. In this example, the sensor apparatus comprises a planar electrode assembly 11 having one or more electrode arrangements on the surface.

In this example, a porous separation membrane 12 is placed in direct contact with and to cover the active electrode area with the dual function of separating the blood sample and directing the transport of separated plasma product to each electrode. Electrical connection is via suitably arranged contacts 16 familiar to those skilled in the art. This enables connection to a measurement device that may implement an appropriate electroanalytical technique, preferably chrono-amperometry.

An electrode assembly holder comprises a bottom plate 14 with a recess to accommodate the electrode assembly 17 and a top plate 13 that also houses a sample entry point 15. This 15 may be designed for an optimum geometry to both measure a pre-set volume of blood and also deliver it for separation and transport by the porous separation membrane 12. The design of the sample entry point may be conical or any other geometric shape that accommodates a pre-determined volume of blood, or it may include the provision of capillary fill geometry. Other physical configurations are also envisaged as is familiar to those skilled in the art.

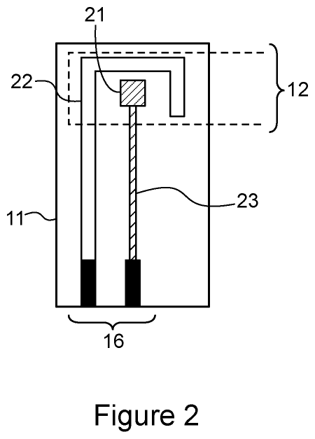

FIG. 2 is a sectional plan view of a single working electrode element of the electrode assembly 11 of the lactate sensor which is fed from a branch of the porous separation membrane 12 which contacts both the working electrode pad 21 and the combined reference/counter (silver/silver chloride) electrode 22. The exact geometry of the working electrode is further defined by the insulated section on the connecting strip 23. This geometry is typical of commercially available screen-printed electrode assemblies. [2] Other physical configurations are also envisaged whereby an array of working electrodes is served by a single combined reference/counter electrode, or a single reference electrode with individual counter electrodes, or a single, but separate counter electrode.

The electrode arrangement is not confined to a planar geometry. It is quite feasible to construct a sandwich arrangement where a working electrode is face-to-face with a reference/counter electrode separated by the porous paper membrane. Concentric tubular geometry may also be implemented. For each geometry, the selection may be based on ease of manufacture for minimisation of unit cost, or to impart a transport advantage through selection of the porous separation membrane, or to enhance electrode sensitivity and response time. Surprisingly, the planar geometry as shown in FIG. 1 is an efficient design that both separates the blood sample and delivers the blood plasma fraction reproducibly to each measurement electrode.

In one example embodiment, the working electrode pad 21 is pre-coated with a phosphate buffer solution (0.05 M, pH 8.0): 6 μL/electrode; with the addition of NAD+: 120 μg/electrode; with the further addition of LDH: 10 units/electrode [816 units/mg]. The porous separation membrane (which for this two-electrode assembly is 27 mm long by 7 mm wide) is fabricated from a qualitative filter paper (circles, diameter: 42.5 mm; limit: 0.22 psi wet burst, 37 sec/100 mL speed (Herzberg); thickness: 205 μm, pore size: 20-25 μm (Particle retention)).

In one example embodiment, only one working electrode 11 is required, leading to a low cost and smaller configuration of the device.

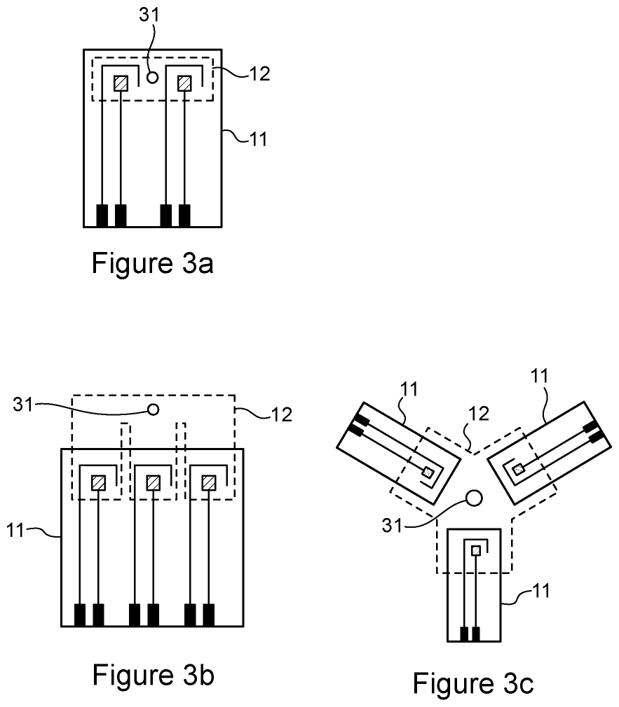

FIG. 3a illustrates another and preferred example; two separate working electrodes are provided, which may allow improved measurements. Suitably, these electrodes are fed from a common liquid sample via the porous separation membrane 12 which acts as a separator and induces transportation to both electrodes from a common sample introduction port 31.

However, several additional and interesting and surprising advantages have now been identified where several electrodes may be employed, particularly in the context of lactate measurement in a blood sample. FIG. 3b is an example embodiment where three electrodes are set in a linear array and fed from a common sampling port 31 via a shaped porous separation membrane 12 separation and transport membrane to provide each electrode with a representative and equivalent sample of blood plasma.

In another example (FIG. 3c), individual electrodes on separate substrates 11 are linked via a common porous separation membrane 12 which is shaped to promote equality in the delivery of blood plasma from a single sample introduction via a central sampling port 31. It may be appreciated that many other specific configurations of the apparatus are also possible. For example additional electrodes may be incorporated and different feed geometries of the porous separation membrane utilised to provide equality or skewed delivery of the blood plasma sample.

In one example, a branch of the porous separation membrane 12 may be pre-loaded with a reagent that affects the measurement at only the electrode associated with that branch. For example, the reagent may be a deliberate addition of a known quantity of lactate to enhance the electrode signal for the purpose of imparting measurement accuracy through calibration.

Other reagents may be employed in order to eliminate interferences from the chemistry of the blood plasma sample in such a way that a correction algorithm may be formulated from the signals of two electrodes, where one electrode experiences the interference and the other does not (due to the reagent addition).

Furthermore, two or more reagents may be added to the porous separation membrane as a sequence through the addition of discrete zones of reagent along the flow pathway of the blood plasma sample. Each zone may be added according to the travel of the blood plasma away from the sampling inlet 31 to the extremity of the initially dry porous separation membrane. Other methods of reagent addition are also envisaged, such as reagent preloading of the electrode surface as is familiar to those skilled in the art.

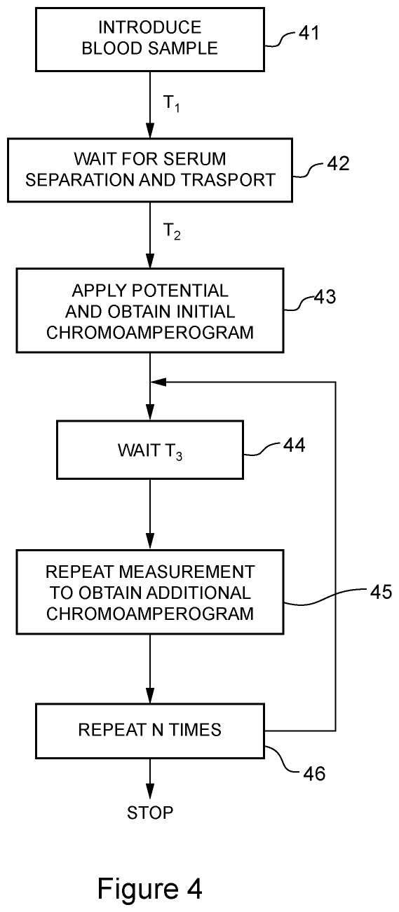

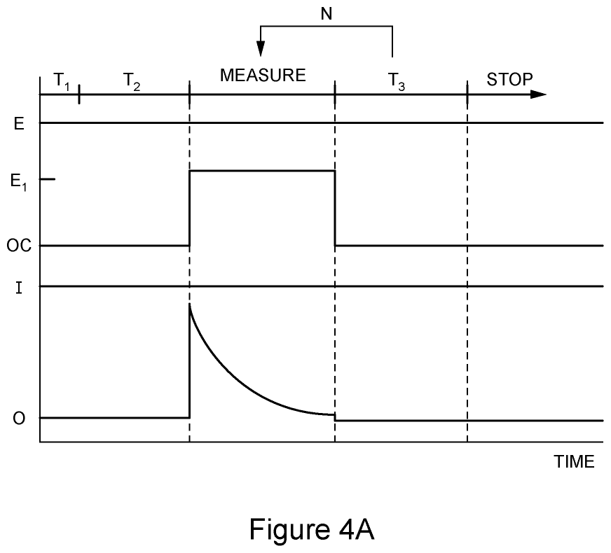

FIG. 4 is a flowchart as a schematic overview of an example method of measuring lactate in blood.

Step 41 comprises the addition of a specified volume of whole blood, either metered by an external device such as a pipette, or through volumetric sampling by the geometry of the sample introduction port 31, for example when configured to operate as a capillary fill sampler. T1 (for example 2 seconds) is the time required for the blood sample to be introduced into the device.

Step 42 comprises the status when the blood plasma has equilibrated and wetted the working electrode(s) surface(s). T2 (for example 2 seconds for a single electrode arrangement, and longer for multiple-electrode assemblies) is a development time to ensure the activation of the enzyme, cofactor and mediator associated with the working electrode and the activation of the reference electrode.

Step 43 is the application of a fixed potential to the working electrode(s) and the initiation of sequential sampled current data acquisition (with a typical scan time of 30 seconds).

Step 44 comprises the conclusion of the chrono-amperometric scan and consolidation of an open circuit, followed by a selected waiting time T3 (for example 1 minute).

Step 45 comprises a repeat chrono-amperometric scan that follows either an identical or different measurement scan to the initial scan.

Step 46 is the repeat of the scan process until sufficient chrono-amperometric data scans have been acquired.

FIG. 4A is a schematic overview of an example method of measuring lactate in blood in terms of operational timing. The three rows represent respectively: The unit operations, previously introduced in FIG. 4; the applied potential to the electrode system, where OC represents a state of Open Circuit, and E1 is the optimised applied potential; and the measured current, where 0 represents the residual background current, close to zero current.

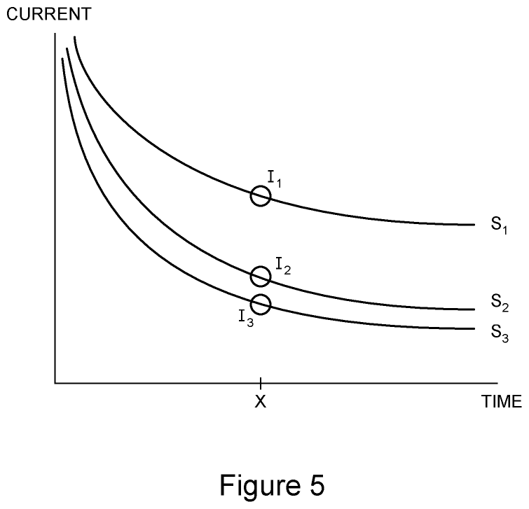

FIG. 5 shows a set of chrono-amperometric scans of the response of any of the electrodes employed in the lactate sensor apparatus. After a selected current decay time X, the first amperometric scan S1 yields a sampled current I1. Subsequent scans, S2 and S3 yield further sampled currents I2 and I3 respectively. In the preferred embodiment, an algorithm may be applied that maximises measurement precision. Surprisingly, the mean of I2 with I3 yields a derived current that enhances measurement precision. Also surprisingly, the difference between the derived current and I1 provides a quality factor that may be used as a threshold against electrode assembly ageing.

It is apparent to persons skilled in the art that there are many permutations and combinations of the chrono-amperometric time sampled current data that may yield a derived current that offers enhanced measurement precision and also yield quality factor data.

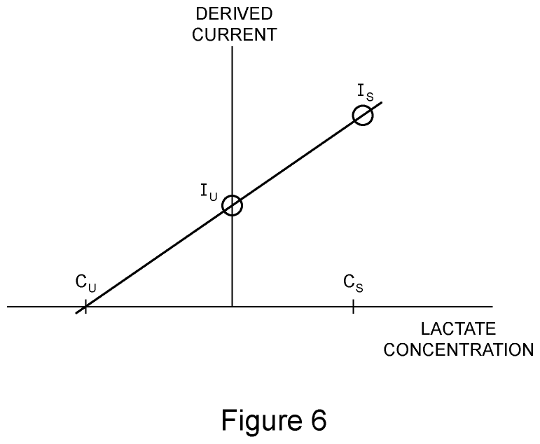

FIG. 6 is a graph that shows the scheme of standard addition calibration for a pair of working electrodes where the second electrode had had the addition of a standard concentration of lactate. One electrode, using the scheme described in FIG. 5, yields the derived current due to the blood plasma alone Iu. The second electrode, also using the scheme described in FIG. 5, due to the additional concentration of lactate present, yields the derived current Is which corresponds to the addition of lactate Cs. Rectilinear construction of the data further yields the concentration Cu of the lactate in the blood plasma alone.

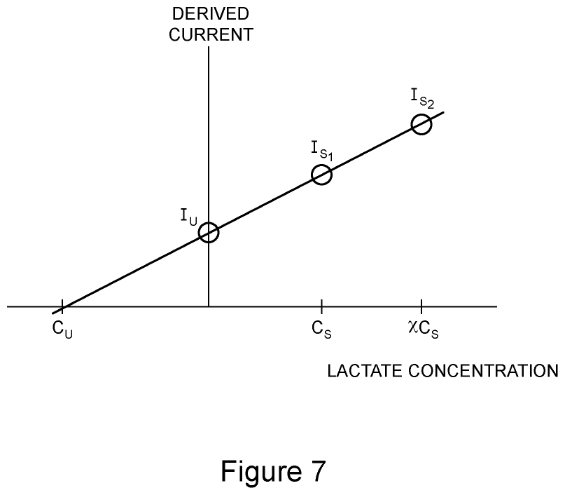

FIG. 7 is a graph that shows the scheme of standard addition calibration for at least three working electrodes where a first electrode yields the derived current due to the blood plasma alone Iu. A second electrode is exposed to the blood plasma sample with the deliberate addition of a known concentration of lactate Cs. This second electrode yields the derived current Is1. A third electrode, similarly, is used to measure the blood plasma sample to which a greater amount of lactate has been added xCs, where x is a number (preferably an integer) greater than 1, with a more preferred value of 2. The third electrode yields the derived current Is2. Rectilinear construction of the three data points Iu, Is1 and Is2 yields the concentration Cu of the lactate in the blood plasma alone. The combination of these three measurements yields greater precision for the determination of the blood plasma lactate concentration Cu than either the single or dual electrode variants.

A further surprising advantage is that the three electrode measurements also supply a linearity quality factor. This provides greater measurement precision where a greater range of blood plasma lactate concentration is encountered. It is apparent to persons skilled in the art that the addition of a greater number of working electrodes and associated standard additions yields increasing data quality through greater precision and a quality factor measurement of higher accuracy.

The selection of the number of electrodes depends upon the immediate application of the measurement. A measurement of lower criticality may require only a “traffic light” output, and a single electrode may be sufficient. As the importance for accuracy and precision increases (such as for critical healthcare), it may be advantageous to use more electrodes and thereby adopt the multi-point standard addition scheme.

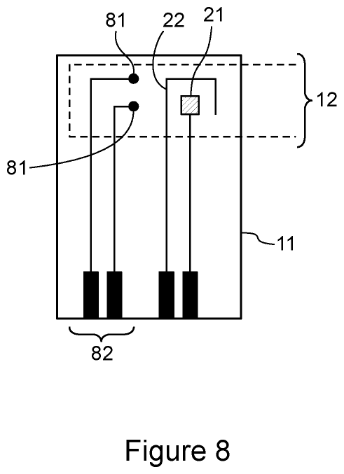

FIG. 8 is a sectional plan view of the lactate sensor electrode assembly, shown for a single working electrode, with a pair of conductivity electrodes 81 and 82. These, placed at the end of the porous paper membrane accurately assess when the blood plasma has been fully transported. They are connected to measurement instrumentation via connectors 82. While adding marginally to the complexity of fabrication, the conductimetric detection of the arrival of the blood plasma front introduces two distinct advantages:

Firstly, it replaces the need for (and improves over) a fixed wait time (T2) for arrival of the blood plasma. This reduces error due to any artefacts of the measurement such as temperature and variation in the paper transport membrane, and also compensates for differences in each blood sample. The conductimetric measurement allows for optimum measurement initiation regardless of experimental variation due to the blood sample and local environmental conditions at which the measurement is made. A second benefit is optimised timing in terms of wait time (T2) minimisation, such that the measurement is made more quickly that using a fixed time for T2.

Additional conductivity sensing electrodes may be placed at other critical positions in the transport geometry to enhance measurement precision through timing the arrival/passing of the sample front.

An example of the conductivity measurement circuit comprises a high impedance (>1MΩ) resistive divider, fed from a constant voltage source, with the conductivity electrodes, electrodes 81, connected to one arm of the divider. A logic circuit, such as a CMOS Schmitt trigger, may be used to monitor for the sudden increase in conductivity associated with the arrival of the blood plasma front on the porous separation membrane adjacent to the conductivity electrodes 81. The circuit generates a digital single bit that indicates when the blood plasma has arrived at the conductivity electrodes, and thereby the adjacent arrival at the working electrode.

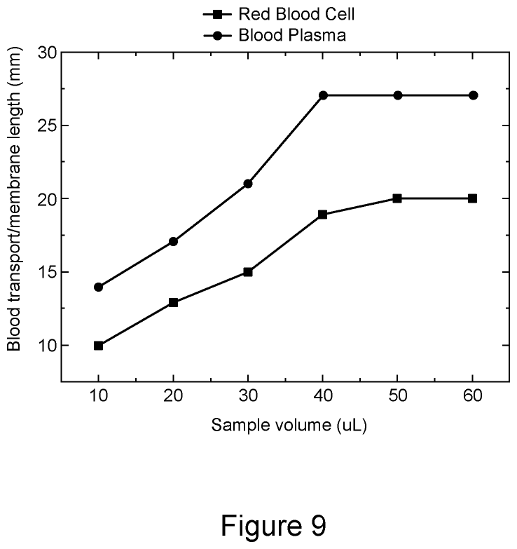

FIG. 9 shows the effective separation of blood plasma from whole blood where a 27 mm×7 mm strip of porous separation membrane has been combined with a similar sized flexible plastic laminate that additionally comprises an entry hole of 4 mm diameter situated centrally and at 4 mm from one end of the 27 mm strip. For all sample volumes greater than 30 μL, the geometric separation is at least 7 mm (which is greater than the longitudinal distance across the working electrode). Even at a sample volume of 10 μL there is a sufficient longitudinal separation of 4 mm.

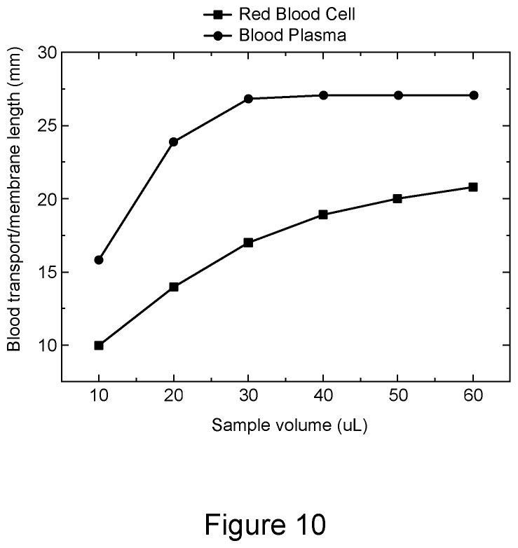

FIG. 10 shows the effective separation of blood plasma from whole blood where a 27 mm×7 mm strip of porous separation membrane has been combined with a similar sized flexible plastic laminate that additionally comprises an entry hole of 4 mm diameter situated centrally along the 27 mm strip, thus allowing bidirectional transport of blood plasma. For all sample volumes the geometric separation is at least 6 mm (which is greater than the longitudinal distance across the working electrode).

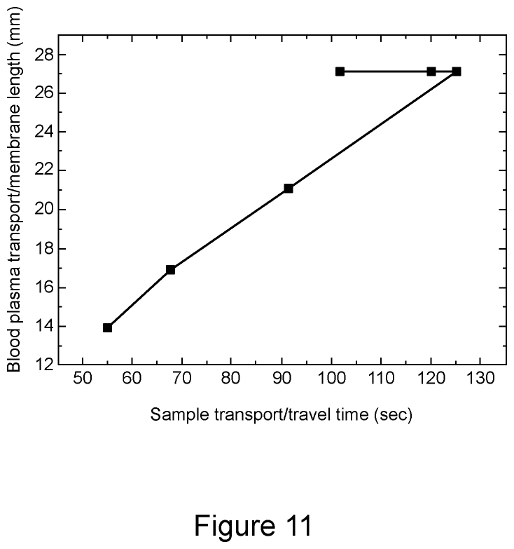

FIG. 11 shows the unidirectional transport time and distance along the separation strip described in FIG. 9. The results are provided below:

| Sample | measurement |
|---|---|
| volume (uL) | time (secs) |
| 10 | 55 |
| 20 | 68 |
| 30 | 92 |
| 40 | 125 |
| 50 | 120 |
| 60 | 102 |

Sample volumes above 30 μL all reach the end of the 27 mm strip.

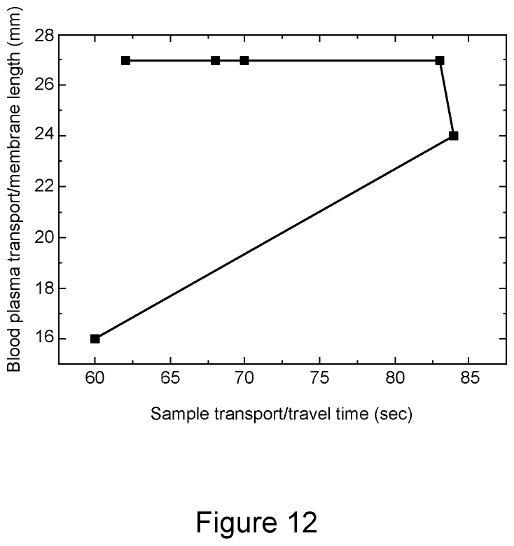

FIG. 12 shows the bidirectional transport time and distance along the separation strip described in FIG. 10. The results are provided below:

| Sample | measurement |
|---|---|
| volume (uL) | time (secs) |
| 10 | 60 |
| 20 | 84 |
| 30 | 83 |
| 40 | 70 |
| 50 | 68 |
| 60 | 62 |

Sample volumes above 20 μL all reach the end of the 27 mm strip. The distance and time displayed are for the total bidirectional distance from the centre inlet point.

Throughout the description and Claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

All documents referred to herein are incorporated by reference.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

### REFERENCES

[1] Electroanalysis 1996, 8, 539
[2] Gwent Electronic Materials Limited—Item Code: BE2031028D1/247

1. A device for measuring a biomarker in a sample consisting of whole blood, comprising:
an entrance for the whole blood sample;
two or more measurement cells, each measurement cell comprising three electrodes, wherein at least one electrode per measurement cell comprises a bio-catalyst specific to the biomarker and at least one electrode per measurement cell comprises an electrochemical mediator; and
a separation means provided between the entrance and the measurement cells;
wherein the separation means is configured to separate whole blood into blood components and is configured to transport at least one of the blood component(s) towards the measurement cells; and
wherein the separation means includes a first pre-determined amount of the biomarker at a region proximate to one of the measurement cells, wherein the region is within the 10% of the length of the separation means closest to the measurement cell and wherein the region is at a distance of more than 15% of the length of the separation means from any of the other measurement cells.

2. The device as claimed in claim 1, wherein the biomarker is lactate.

3. The device as claimed in claim 1, wherein the separation means includes paper having a porosity of at least 2%.

4. The device as claimed in claim 1, wherein the separation means includes a composite comprising paper and a material with a lower porosity than paper.

5. (canceled)

6. (canceled)

7. The device as claimed in claim 1 comprising at least three measurement cells wherein the separation means includes a second pre-determined amount of the biomarker at a region proximate to one of the measurement cells,
wherein the region is within the 10% of the length of the separation means closest to the measurement cell and wherein the region is at a distance of more than 15% of the length of the separation means from any of the other measurement cells,
wherein the first pre-determined amount of the biomarker differs from the second pre-determined amount of the biomarker.

8. The device as claimed in claim 7, wherein the region proximate to the measurement cell is spaced away from the entrance by a distance corresponding to more than 10% of the length of the separation means and the whole blood sample does not contact the first pre-determined amount of the biomarker or the second pre-determined amount of the biomarker.

9. The device as claimed in claim 1, wherein the second measurement cell includes a third pre-determined amount of the biomarker.

10. The device as claimed in claim 7 comprising at least three measurement cells wherein the third measurement cell includes a fourth pre-determined amount of the biomarker and the first pre-determined amount of the biomarker differs from the fourth pre-determined amount of the biomarker.

11. The device as claimed in claim 1, wherein the separation means has an associated porosity of 2%-20%, and/or the pores formed within the separation mean have a mean diameter of 2 to 40 μm.

12. The device as claimed in claim 1, wherein the separation means comprises two or more sheets of material and at least one of the sheets has a porosity of at least 4% and/or a pore size of 2 to 10 μm.

13. (canceled)

14. (canceled)

15. The device as claimed in claim 1, wherein the separation means extends from the entrance to the measurement cell(s), or to within 5 mm of each of the measurement cells.

16. The device as claimed in claim 1, including at least four measurement cells, wherein the third measurement cell includes a bio-catalyst specific to a second bio-marker and the fourth measurement cell includes a bio-catalyst specific to a second bio-marker and a pre-determined amount of the second biomarker.

17. The device of claim 4 wherein the separation means is configured to allow blood sample to be transferred from the entrance to each measurement cell, and the separation means is configured to resist or prevent transfer of red blood cells to the measurement cells.

18. (canceled)

19. The device as claimed claim 4 wherein the measurement cells are arranged linearly or to radially extend from the entrance.

20. (canceled)

21. The device as claimed in claim 1, wherein the device is an amperometric biosensor.

22. The device as claimed in claim 1, comprising one or more conductivity electrodes configured to detect the arrival of liquid sample.

23. A method of measuring a biomarker in whole blood comprising:
providing the device as claimed in claim 1;
introducing a whole blood sample through the entrance of the device;
allowing the separation means to separate the whole blood into its components;
obtaining a measurement from the measurement cells;
wherein the measurement is obtained within 5 minutes of the sample being introduced through the entrance.

24. The method as claimed in claim 23, wherein measurements are made simultaneously from each of the measurement cells.

25. The method as claimed in claim 23, wherein multiple measurements are made from each measurement cell at precisely selected time intervals.

26. The device as claimed in claim 23, wherein the biomarker is lactate.
